(19) United States
(12) Patent Application Publication
(10) Pub. No.: US 2008/0131475 A1
(43) Pub. Date: Jun. 5, 2008

(54) Methods of Use of Dual Ppar Agonist Compounds and Drug Delivery Devices Containing Such Compounds

(76) Inventor: David Saul Cohen, Summit, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/814,265

(22) PCT Filed: Jan. 17, 2006
(86) PCT No.: PCT/US06/01506

§ 371 Date: Jul. 18, 2007

Related U.S. Application Data
(60) Provisional application No. 60/644,646, filed on Jan. 18, 2005.

(51) Int. Cl. A61F 2/04; A61P 29/00; A61P 9/00; A61K 31/397; A61K 31/40; A61K 31/421; A61K 31/404

(52) U.S. Cl. 424/423; 514/210.17; 514/423; 514/419; 514/210.18; 514/374

(57) ABSTRACT

The present invention relates to uses of dual PPAR agonist compounds and delivery devices containing such compounds. The compounds are useful as pharmaceuticals for the treatment and/or prevention of VSMC proliferation, e.g., stenosis and restenosis, especially restenosis in diabetic patients.

Many humans suffer from circulatory diseases caused by progressive blockade of the blood vessels that perfuse the heart and other major organs. Severe blockage of blood vessels in such humans often leads to ischemic injury, stroke, myocardial infarction or congestive heart failure. Atherosclerotic lesions which limit or obstruct coronary or peripheral blood flow are the major causes of ischemic disease related morbidity and mortality including coronary heart disease and stroke. To stop the disease process and prevent the more advanced disease states in which the cardiac muscle or other organs are compromised, medical revascularization procedures, such as percutaneous transluminal coronary angioplasty (PCTA), percutaneous transluminal angioplasty (PTA) with stent placement, atherectomy, bypass grafting or other types of vascular grafting procedures are used.

Re-narrowing (restenosis in diabetics and non-diabetics) of an artherosclerotic coronary artery after various revascularization procedures occurs in 10-80% of patients undergoing this treatment, depending on the procedure used and the arterial site. Besides opening an artery obstructed by atherosclerosis, revascularization also injures the luminal endothelial cell lining and smooth muscle cells within the vessel wall, thus initiating a thrombotic and inflammatory response. Cell derived growth factors such as platelet derived growth factor, infiltrating macrophages, leukocytes or the smooth muscle cells themselves provoke proliferative and migratory responses in the smooth muscle cells. Simultaneous with local proliferation and migration, inflammatory cells also invade the site of vascular injury and may migrate to the deeper layers of the vessel wall. Proliferation/migration usually begins within one to two days post-injury and, depending on the revascularization procedure used, continues for days and weeks. Vascular stenosis and re-canalization-induced restenosis is a particularly acute problem in diabetics, in particular, insulin dependent diabetics.

Following luminal expansion, cells within the atherosclerotic lesion and media migrate, proliferate and/or secrete significant amounts of extracellular matrix proteins. Proliferation, migration and extracellular matrix synthesis continues until the damaged endothelial layer is repaired at which time proliferation slows within the intima. The newly formed tissue is called neointima, intimal thickening or restenotic lesion and usually results in narrowing of the vessel lumen. Further lumen narrowing may take place due to constructive remodeling, e.g., vascular remodeling, leading to further intimal thickening or hyperplasia.

Moreover, there are also atherosclerotic lesions which do not limit or obstruct vessel blood flow but which form the so-called “vulnerable plaques”. Such atherosclerotic lesions or vulnerable plaques are prone to rupture or ulceration, which results in thrombosis and can produce unstable angina pectoris, myocardial infarction or sudden death. Inflamed atherosclerotic plaques can be detected by thermography.

Peroxisome proliferator receptors (PPAR) agonists are implicated in a number of biological processes and disease states including hypercholesterolemia, hyperlipidemia and diabetes. PPARs are members of the nuclear receptor superfamily of transcription factors that includes steroid, thyroid and vitamin D receptors. They play a role in controlling expression of proteins that regulate lipid metabolism. Furthermore, the PPARs are activated by fatty acids and fatty acid metabolites. There are three PPAR subtypes PPARα, PPARβ (also referred to as PPARδ) and PPARγ. Each receptor shows a different pattern of tissue expression, and differences in activation by structurally diverse compounds. PPARγ, for instance, is expressed most abundantly in adipose tissue and at lower levels in skeletal muscle, heart, liver, intestine, kidney, vascular endothelial and smooth muscle cells and macrophages. PPAR receptors are associated with regulation of insulin sensitivity and blood glucose levels, macrophage differentiation, inflammatory responses and cell differentiation. Accordingly, PPARs have been associated with obesity, diabetes, carcinogenesis, the hyperplasia associated with atherosclerosis, hyperlipidemia and hypercholesterolemia.

With respect to vascular smooth cell proliferative diseases or disorders, there are conflicting hypotheses with respect to PPAR agonists. Some references show that selective PPARγ agonists may protect the vasculature from diabetes-enhanced injury because they are potent inhibitors of vascular smooth muscle cell (VSMC) migration pathways. See Goetze et al., J Cardiovasc Pharmacol, Vol. 33, No. 5, pp. 798-806 (1999). However, PPAR delta has been implicated in playing an important role in the pathology of diseases associated with VSMC proliferation, such as primary atherosclerosis and restenosis, since overexpression of PPARδ in VSMC increased post-confluent cell proliferation by increasing the cyclin A and CDK2 as well as decreasing p57(kip2). See Zhang et al., J Biol Chem, Vol. 277, No. 13, pp. 11505-11512 (2002). Moreover, in another study comparing PPARγ ligands to PPARα ligands, the PPARγ ligand rosiglitazone, was found to decrease intimal hyperplasia following balloon injury in both fatty and lean Zucker rats but not the PPARα ligand fenofibrate. Accordingly, PPAR agonist compounds are still considered to have non-uniform effectiveness or, in the case of some PPAR agonists, no effect at all on VSMC proliferative diseases or disorders or actually to be the cause of these diseases or disorders.

For the reasons set forth above, there is a need for dual PPARα/γ agonists that can be used alone or in combination to treat and/or prevent VSMC diseases or disorders.

In one aspect, the present invention provides a method of treating and/or preventing VSMC proliferative diseases or disorders comprising administering a therapeutically effective amount of a dual PPARα/γ agonist compound or a pharmaceutically acceptable salt thereof to a mammal in need thereof.

In yet another aspect of the present invention, there is provided a drug delivery device for local administration of a therapeutically effective amount of a dual PPARα/γ agonist compound or a pharmaceutically acceptable salt thereof for treating and/or preventing VSMC proliferative diseases or disorders.

In still another aspect of the present invention, there is provided a method of treating and/or preventing VSMC proliferative diseases or disorders comprising locally administering via a drug delivery device a therapeutically effective amount of a dual PPARα/γ agonist compound or a pharmaceutically acceptable salt thereof to a mammal in need thereof.

In a preferred embodiment, the drug delivery device is a stent.

In another aspect of the present invention, there is provided a method of treating and/or preventing VSMC proliferative diseases or disorders comprising administering a therapeutically effective amount of a dual PPARα/γ agonist compound or a pharmaceutically acceptable salt thereof in combination with another therapeutic agent.

In a preferred embodiment, the VSMC proliferative diseases or disorders, as mentioned herein, are ureteral and/or biliary proliferation, and coronary artery and peripheral arterial stenosis and restenosis in diabetics and non-diabetics.

As described above, the present invention provides a method of treating and/or preventing VSMC proliferative diseases or disorders comprising administering a therapeutically effective amount of a dual PPARα/γ agonist compound or a pharmaceutically acceptable salt thereof to a mammal in need thereof.

It has surprisingly been found that dual PPARα/γ agonist compounds markedly reduce or even prevent VSMC proliferation and therefore may be employed in the treatment of diseases or disorders wherein VSMC proliferation is an underlying cause of the disease or disorder. For example, a dual PPARα/γ compound may be employed to treat the occurrence of vascular stenosis and restenosis in mammals, particularly humans, and preferably in those who are diabetics.

In a preferred embodiment of this aspect of the invention, the dual acting PPARα/γ agonists within the scope of this invention include, but are not limited to, compounds of formula (I)

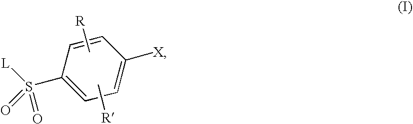

wherein
L is

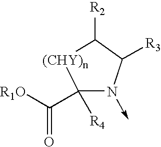

radical,
in which
   R₁ is hydrogen, optionally substituted alkyl, aryl, heteroaryl, aralkyl or cycloalkyl;
   R₂ is hydrogen, hydroxy, optionally substituted alkyl, aryl, aralkyl, alkoxy, aryloxy, aralkoxy, alkylthio, arylthio or aralkylthio;
   R₃ is hydrogen or aryl, or
   R₂ and R₃ combined are alkylene which together with the carbon atoms they are attached to form a 5- to 7-membered ring;
   n is zero or an integer from 1-2;
   Y is hydrogen, or
   Y and R₂, taken together with the carbon atoms they are attached to, form a bond provided that n is 1;
   R₄ is hydrogen, or
   R₄ and Y, taken together with the carbon atoms they are attached to, form a bond provided that n is 1, and
   R₂ and R₃, taken together with the carbon atoms they are attached to, form a bond, or
L is

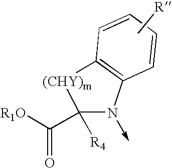

radical,
in which
   R₁ is hydrogen, optionally substituted alkyl, aryl, heteroaryl, aralkyl or cycloalkyl;
   R″ is hydrogen, optionally substituted alkyl, alkoxy or halogen;
   m is an integer from 1-2;
   Y is hydrogen;
   R₄ is hydrogen, or
   R₄ and Y, taken together with the carbon atoms they are attached to, form a bond provided that m is 1;
R and R′ are, independently, hydrogen, halogen, optionally substituted alkyl, alkoxy, aralkyl or heteroaralkyl, or
R and R′, combined together, form a methylenedioxy group provided that R and R′ are attached to carbon atoms adjacent to each other, or
R and R′, combined together with the carbon atoms they are attached to, form an optionally substituted 5- to 6-membered aromatic or heteroaromatic ring provided that R and R′ are attached to carbon atoms adjacent to each other, or
R—C and R′—C may independently be replaced by nitrogen;
X is —Z—(CH₂)ₚ—Q—W,
wherein
   Z is a bond, O, S, S(O), S(O)₂, —C(O)— or —C(O)NR₅—, in which R₅ is hydrogen, alkyl or aralkyl;
   p is an integer from 1-8;
   Q is a bond provided that Z is not a bond when p is 1, or
   Q is —O(CH₂)ᵣ— or —S(CH₂)ᵣ— in which r is zero or an integer from 1-8, or
   Q is —O(CH₂)₁₋₈O—, —S(CH₂)₁₋₈O—, —S(CH₂)₁₋₈S—, —C(O)— or —C(O)NR₆— in which R₆ is hydrogen, optionally substituted alkyl, cycloalkyl, aryl, heteroaryl, aralkyl or heteroaralkyl, or
   Q is —NR₆—, —NR₅C(O)—, —NR₅C(O)NH— or —NR₅C(O)O— provided that p is not 1;
   W is cycloalkyl, aryl, heterocyclyl, aralkyl or heteroaralkyl, or
   W and R₆, taken together with the nitrogen atom to which they are attached, form a 8- to 12-membered bicyclic ring, which may be optionally substituted or may contain another heteroatom selected from oxygen, nitrogen and sulfur;
      or a pharmaceutically acceptable salt thereof; or an optical isomer thereof; or a mixture of optical isomers thereof.

Listed below are definitions of various terms used to describe the compounds of the instant invention. These definitions apply to the terms as they are used throughout the specification and the claims unless they are otherwise limited in specific instances either individually or as part of a larger group, e.g., wherein an attachment point of a certain group is limited to a specific atom within that group, the point of attachment is defined by an arrow at the specific atom.

The term “optionally substituted alkyl” refers to unsubstituted or substituted straight- or branched-chain hydrocarbon groups having 1-20 carbon atoms, preferably 1-7 carbon atoms. Exemplary unsubstituted alkyl groups include methyl, ethyl, propyl, isopropyl, n-butyl, t-butyl, isobutyl, pentyl, hexyl, isohexyl, heptyl, 4,4-dimethylpentyl, octyl and the like. Substituted alkyl groups include, but are not limited to, alkyl groups substituted by one or more of the following groups: halo, hydroxy, cycloalkyl, alkanoyl, alkoxy, alkyloxyalkoxy, alkanoyloxy, amino, alkylamino, dialkylamino, alkanoylamino, thiol, alkylthio, alkylthiono, alkylsulfonyl, arylsulfonyl, heteroarylsulfonyl, sulfonamido, nitro, cyano, carboxy, alkoxycarbonyl, aryl, alkenyl, alkynyl, aralkoxy, guanidino, heterocyclyl including indolyl, imidazolyl, furyl, thienyl, thiazolyl, pyrrolidyl, pyridyl, pyrimidyl, piperidyl, morpholinyl and the like.

The term “lower alkyl” refers to those alkyl groups as described above having 1-7 carbon atoms, preferably 1-4 carbon atoms.

The term “halogen” or “halo” refers to fluorine, chlorine, bromine and iodine.

The term “alkenyl” refers to any of the above alkyl groups having at least 2 carbon atoms and further containing a carbon to carbon double bond at the point of attachment. Groups having 2-4 carbon atoms are preferred.

The term “alkynyl” refers to any of the above alkyl groups having at least 2 carbon atoms and further containing a carbon to carbon triple bond at the point of attachment. Groups having 2-4 carbon atoms are preferred.

The term “alkylene” refers to a straight-chain bridge of 1-6 carbon atoms connected by single bonds, e.g., —(CH₂)ₓ—, wherein x is 1-6, which may be substituted with 1-3 lower alkyl or alkoxy groups.

The term “cycloalkyl” refers to optionally substituted monocyclic, bicyclic or tricyclic hydrocarbon groups of 3-12 carbon atoms, each of which may optionally be substituted by one or more substituents, such as alkyl, halo, oxo, hydroxy, alkoxy, alkanoyl, amino, alkylamino, dialkylamino, thiol, alkylthio, nitro, cyano, carboxy, carboxyalkyl, alkoxycarbonyl, alkyl- and arylsulfonyl, sulfonamido, heterocyclyl and the like.

Exemplary monocyclic hydrocarbon groups include, but are not limited to, cyclopropyl, cyclobutyl, cyclopentyl, cyclopentenyl, cyclohexyl and cyclohexenyl and the like.

Exemplary bicyclic hydrocarbon groups include bornyl, indyl, hexahydroindyl, tetrahydronaphthy 1, decahydronapht hyl, bicyclo[2.1.1]hexyl, bicyclo[2.2.1]heptyl, bicyclo[2.2.1]heptenyl, 6,6-dimethylbicyclo[3.1.1]heptyl, 2,6,6-trimethylbicyclo[3.1.1]heptyl, bicyclo[2.2.2]octyl and the like.

Exemplary tricyclic hydrocarbon groups include adamantyl and the like.

The term “alkoxy” refers to alkyl-O—.

The term “acyl” refers to alkanoyl, aroyl, heteroaroyl, arylalkanoyl or heteroarylalkanoyl.

The term “alkanoyl” refers to alkyl-C(O)—.

The term “alkanoyloxy” refers to alkyl-C(O)—O—.

The terms “alkylamino” and “dialkylamino” refer to alkyl-NH— and (alkyl)₂N—, respectively.

The term “alkanoylamino” refers to alkyl-C(O)—NH—.

The term “alkylthio” refers to alkyl-S—.

The term “alkylaminothiocarbonyl” refers to alkyl-NHC(S)—.

The term “trialkylsilyl” refers to (alkyl)₃Si—.

The term “trialkylsilyloxy” refers to (alkyl)₃SiO—.

The term “alkylthiono” refers to alkyl-S(O)—.

The term “alkylsulfonyl” refers to alkyl-S(O)₂—.

The term “alkoxycarbonyl” refers to alkyl-O—C(O)—.

The term “alkoxycarbonyloxy” refers to alkyl-O—C(O)O—.

The term “carbamoyl” refers to alkyl-NHC(O)—, (alkyl)₂NC(O)—, aryl-NHC(O)—, alkyl(aryl)-NC(O)—, heteroaryl-NHC(O)—, alkyl(heteroaryl)-NC(O)—, aralkyl-NHC(O)— and alkyl(aralkyl)-NC(O)—.

The term “aryl” refers to monocyclic or bicyclic aromatic hydrocarbon groups having 6-12 carbon atoms in the ring portion, such as phenyl, naphthyl, tetrahydronaphthyl, biphenyl and diphenyl groups, each of which may optionally be substituted by 1-4 substituents, such as alkyl, halo, hydroxy, alkoxy, alkanoyl, alkanoyloxy, optionally substituted amino, thiol, alkylthio, nitro, cyano, carboxy, carboxyalkyl, alkoxycarbonyl, alkylthiono, alkyl- and arylsulfonyl, sulfonamido, heterocycloyl and the like.

The term “monocyclic aryl” refers to optionally substituted phenyl as described under aryl.

The term “aralkyl” refers to an aryl group bonded directly through an alkyl group, such as benzyl.

The term “aralkylthio” refers to aralkyl-S—.

The term “aralkoxy” refers to an aryl group bonded directly through an alkoxy group.

The term “arylsulfonyl” refers to aryl-S(O)₂—.

The term “arylthio” refers to aryl-S—.

The term “aroyl” refers to aryl-C(O)—.

The term “aroylamino” refers to aryl-C(O)—NH—.

The term “aryloxycarbonyl” refers to aryl-O—C(O)—.

The term “heterocyclyl” or “heterocyclo” refers to an optionally substituted, fully saturated or unsaturated, aromatic or non-aromatic cyclic group, e.g., which is a 4- to 7-membered monocyclic, 7- to 12-membered bicyclic, or 10- to 15-membered tricyclic ring system, which has at least one heteroatom in at least one carbon atom-containing ring. Each ring of the heterocyclic group containing a heteroatom may have 1, 2 or 3 heteroatoms selected from nitrogen atoms, oxygen atoms and sulfur atoms, where the nitrogen and sulfur heteroatoms may also optionally be oxidized. The heterocyclic group may be attached at a heteroatom or a carbon atom.

Exemplary monocyclic heterocyclic groups include pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, 4-piperidonyl, pyridyl, pyrazinyl, pyrimidinyl, pyridazinyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl and the like.

Exemplary bicyclic heterocyclic groups include indolyl; dihydroidolyl; benzothiazolyl; benzoxazinyl; benzoxazolyl; benzothienyl; benzothiazinyl; quinuclidinyl; quinolinyl; tetrahydroq uinolinyl; decahydroq uinolinyl; isoq uinolinyl; tetrahydroisoquinolinyl; decahydroisoquinolinyl; benzimidazolyl; benzopyranyl; indolizinyl; benzofuryl; chromonyl; coumarinyl; benzopyranyl; cinnolinyl; quinoxalinyl; indazolyl; pyrrolopyridyl; furopyridinyl, such as furo[2,3-c]pyridinyl, furo[3,2-b]-pyridinyl] or furo[2,3-b]pyridinyl; dihydroisoindolyl; dihydroquinazolinyl, such as 3,4-dihydro-4-oxo-quinazolinyl; phthalazinyl; and the like.

Exemplary tricyclic heterocyclic groups include carbazolyl, dibenzoazepinyl, dithienoazepinyl, benzindolyl, phenanthrolinyl, acridinyl, phenanthridinyl, phenoxazinyl, phenothiazinyl, xanthenyl, carbolinyl and the like.

The term “heterocyclyl” includes substituted heterocyclic groups. Substituted heterocyclic groups refer to heterocyclic groups substituted with 1, 2 or 3 of the following:
(a) alkyl;
(b) hydroxy (or protected hydroxy);
(c) halo;
(d) oxo, i.e., =O;
(e) optionally substituted amino, alkylamino or dialkylamino;
(f) alkoxy;
(g) cycloalkyl;
(h) carboxy;
(i) heterocyclooxy;
(j) alkoxycarbonyl, such as unsubstituted lower alkoxycarbonyl;
(k) mercapto;
(l) nitro;
(m) cyano;
(n) sulfonam ido, sulfonam idoalkyl, sulfonam idoaryl or sulfonam idodialkyl;
(o) aryl;
(p) alkylcarbonyloxy;
(q) arylcarbonyloxy;
(R) arylthio;
(s) aryloxy;
(t) alkylthio;
(u) formyl;
(v) carbamoyl;
(w) aralkyl; or
(x) aryl substituted with alkyl, cycloalkyl, alkoxy, hydroxy, amino, alkylamino, dialkylamino or halo.

The term “heterocyclooxy” denotes a heterocyclic group bonded through an oxygen bridge.

The term “heteroaryl” refers to an aromatic heterocycle, e.g., monocyclic or bicyclic aryl, such as pyrrolyl, pyrazolyl, imidazolyl, oxazolyl, isoxazolyl, thiazolyl, isothiazolyl, furyl, thienyl, pyridyl, pyrazinyl, pyrimidinyl, pyridazinyl, indolyl, benzothiazolyl, benzoxazolyl, benzothienyl, quinolinyl, isoquinolinyl, benzimidazolyl, benzofuryl and the like; optionally substituted by, e.g., lower alkyl, lower alkoxy or halo.

The term “heteroarylsulfonyl” refers to heteroaryl-S(O)₂—.

The term “heteroaroyl” refers to heteroaryl-C(O)—.

The term “heteroaralkyl” refers to a heteroaryl group bonded through an alkyl group.

Encompassed by the invention are prodrug derivatives, e.g., any pharmaceutically acceptable prodrug ester derivatives of the carboxylic acids of the invention which are convertible by solvolysis or under physiological conditions to the free carboxylic acids.

Examples of such carboxylic acid esters are preferably lower alkyl esters, cycloalkyl esters, lower alkenyl esters, benzyl esters, mono or disubstituted lower alkyl esters, e.g., the ω-(amino, mono- or di-lower alkylamino, carboxy, lower alkoxycarbonyl)-lower alkyl esters, the α-(lower alkanoyloxy, lower alkoxycarbonyl or di-lower alkylaminocarbonyl)-lower alkyl esters, such as the pivaloyloxy-methyl ester, and the like conventionally used in the art.

The compounds of the invention depending on the nature of the substituents, may possess one or more asymmetric centers. The resulting diastereoisomers, optical isomers, i.e., enantiomers, and geometric isomers are encompassed by the instant invention.

Preferred are compounds of formula (I), wherein
X is —Z—(CH₂)ₚ—Q—W,
wherein
   Z is a bond, O, S, —C(O)— or —C(O)NR₅— in which R₅ is hydrogen, alkyl or aralkyl;
   p is an integer from 1-8;
   Q is a bond provided that Z is not a bond when p is 1, or
   Q is —O(CH₂)ᵣ— or —S(CH₂)ᵣ—, in which r is zero or an integer from 1-8, or
   Q is —O(CH₂)₁₋₈O—, —S(CH₂)₁₋₈O—, —S(CH₂)₁₋₈S—, —C(O)— or —C(O)NR₆—, in which R₆ is hydrogen, optionally substituted alkyl, cycloalkyl, aryl, heteroaryl, aralkyl or heteroaralkyl, or
   Q is —NR₆—, —NR₆C(O)—, —NR₅C(O)NH— or —NR₅C(O)O— provided that p is not 1;
   W is cycloalkyl, aryl, heterocyclyl, aralkyl or heteroaralkyl, or
   W and R₆, taken together with the nitrogen atom to which they are attached, form a 8- to 12-membered bicyclic ring, which may be optionally substituted or may contain another heteroatom selected from oxygen, nitrogen and sulfur;
      or a pharmaceutically acceptable salt thereof; or an optical isomer thereof; or a mixture of optical isomers thereof.

Further preferred are compounds of formula (IA),

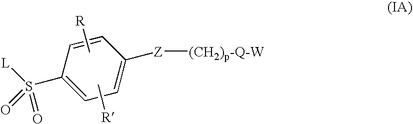

wherein
L is

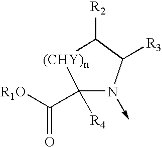

radical,
in which
   R₁ is hydrogen or optionally substituted alkyl;
   R₂ and R₃ are hydrogen, or
   R₂ and R₃ combined are alkylene which, together with the carbon atoms they are attached to, form a 6-membered ring;
   n is zero or an integer from 1-2;
   Y is hydrogen; and
   R₄ is hydrogen, or
L is

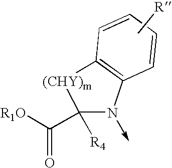

radical,
in which
   R₁ is hydrogen or optionally substituted alkyl;
   R″ is hydrogen, optionally substituted alkyl, alkoxy or halogen;
   m is an integer from 1-2;
   Y is hydrogen; and
   R₄ is hydrogen;
R and R′ are, independently, hydrogen, halogen, optionally substituted C₁₋₆ alkyl or C₁₋₆alkoxy, or
R and R′, combined together, form a methylenedioxy group provided that R and R′ are attached to carbon atoms adjacent to each other;
Z is a bond, O, S or —C(O)NR₅—, in which R₅ is hydrogen, alkyl or aralkyl;
p is an integer from 1-5;
Q is a bond provided that Z is not a bond when p is 1, or
Q is —O(CH₂)ᵣ— or —S(CH₂)ᵣ— in which r is zero, or
Q is —C(O)— or —C(O)NR₆—, in which R₆ is hydrogen, optionally substituted alkyl, cycloalkyl, aryl, heteroaryl, aralkyl or heteroaralkyl, or
Q is —NR₆—, —NR₅C(O)—, —NR₅C(O)NH— or —NR₅C(O)O— provided that p is not 1;
W is cycloalkyl, aryl or heterocyclyl, or
W and R₆, taken together with the nitrogen atom to which they are attached, form a 9- to 10-membered bicyclic ring, which may be optionally substituted or may contain another heteroatom selected from oxygen, nitrogen and sulfur;
   or a pharmaceutically acceptable salt thereof; or an optical isomer thereof; or a mixture of optical isomers thereof.

More preferred are the compounds of formula (IA), wherein
L is

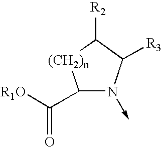

radical,
in which
   R₁ is hydrogen or optionally substituted alkyl;
   R₂ and R₃ are hydrogen; and
   n is zero or an integer from 1-2, or
L is

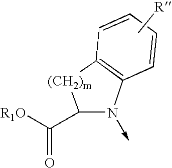

radical,
in which
   R₁ is hydrogen or optionally substituted alkyl;
   R″ is hydrogen; and
   m is an integer from 1 to 2;
R is hydrogen, halogen, optionally substituted C₁₋₆alkyl or C₁₋₆alkoxy;
R′ is hydrogen;
Z is a bond, O or S;
p is an integer from 1-5;
Q is a bond provided that Z is not a bond when p is 1, or
Q is O, S or —C(O)NR₆—, in which R₆ is hydrogen, optionally substituted alkyl or cycloalkyl, or
Q is —NR₆—, —NR₅C(O)NH— or —NR₅C(O)O—, in which R₅ is hydrogen, alkyl or aralkyl provided that p is not 1;
W is cycloalkyl, aryl or heterocyclyl, or
W and R₆, taken together with the nitrogen atom to which they are attached, form a 9- to 10-membered bicyclic ring, which may be optionally substituted or may contain another heteroatom selected from oxygen, nitrogen and sulfur;
   or a pharmaceutically acceptable salt thereof; or an optical isomer thereof; or a mixture of optical isomers thereof.

Most preferred are the compounds of formula (IB)

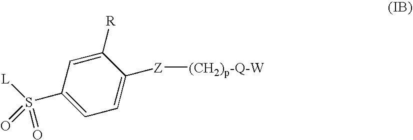

wherein,
L is

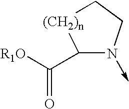

radical,
in which
   R₁ is hydrogen or optionally substituted alkyl; and
   n is zero or 1, or
L is

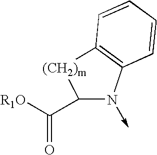

radical,
in which
   R₁ is hydrogen or optionally substituted alkyl; and
   m is 1;
R is hydrogen, halogen, optionally substituted C₁₋₆alkyl or C₁₋₆alkoxy;
Z is a bond, O or S;
p is an integer from 1-5;
Q is a bond provided that Z is not a bond when p is 1, or
Q is O, S or —C(O)NR₆—, in which R₆ is hydrogen, optionally substituted alkyl or cycloalkyl, or
Q is —NR₆—, —NR₅C(O)NH— or —NR₅C(O)O—, in which R₅ is hydrogen, alkyl or aralkyl provided that p is not 1;
W is cycloalkyl, aryl or heterocyclyl, or
W and R6, taken together with the nitrogen atom to which they are attached, form a 9- to 10-membered bicyclic ring, which may be optionally substituted or may contain another heteroatom selected from oxygen, nitrogen and sulfur;
   or a pharmaceutically acceptable salt thereof; or an optical isomer thereof; or a mixture of optical isomers thereof.

Further preferred are compounds of formula (IB), wherein
L is

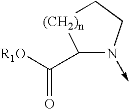

radical,
in which
   R₁ is hydrogen; and
   n is zero or 1;
R is hydrogen, halogen, optionally substituted C₁₋₆alkyl or C₁₋₆alkoxy;
Z is a bond, O or S;
p is an integer from 1-4;
Q is a bond provided that Z is not a bond when p is 1, or
Q is O or S;
W is aryl or heterocyclyl;
   or a pharmaceutically acceptable salt thereof; or an optical isomer thereof; or a mixture of optical isomers thereof.

Further preferred are also the compounds of formula (IB), wherein
L is

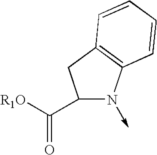

radical, in which R₁ is hydrogen;
R is hydrogen, halogen, optionally substituted C₁₋₆alkyl or C₁₋₆alkoxy;
Z is a bond, O or S;
p is an integer from 1-4;
Q is a bond provided that Z is not a bond when p is 1, or
Q is O or S;
W is aryl or heterocyclyl;
   or a pharmaceutically acceptable salt thereof; or an optical isomer thereof; or a mixture of optical isomers thereof.

Further preferred are also the compounds of formula (IB), wherein the asymmetric center in radical L is in the (R) configuration; or a pharmaceutically acceptable salt thereof.

Further preferred are also the compounds of formula (IB), designated as the A group, wherein
R₁ is hydrogen or optionally substituted alkyl;
R is hydrogen, halogen, optionally substituted C₁₋₆alkyl or C₁₋₆alkoxy;
Z is O or S;
p is 2;
Q is a —NR₆—, in which R₆ is lower alkyl;
W is aryl or heterocyclyl;
   or a pharmaceutically acceptable salt thereof; or an optical isomer thereof; or a mixture of optical isomers thereof.

Preferred are the compounds in the A group, wherein
R is hydrogen, chloro, n-propyl or methoxy;
   or a pharmaceutically acceptable salt thereof; or an optical isomer thereof; or a mixture of optical isomers thereof.

Further preferred are also the compounds of formula (IB), designated as the B group, wherein
R₁ is hydrogen or optionally substituted alkyl;
R is hydrogen, halogen, optionally substituted C₁₋₆alkyl or C₁₋₆alkoxy;
Z is a bond;
p is 2;
Q is a —C(O)NR₆—, in which R₆ is optionally substituted alkyl;
W is aryl or heterocyclyl, or
W and R₆, taken together with the nitrogen atom to which they are attached, form a 9- to 10-membered bicyclic ring, which may be optionally substituted or may contain another heteroatom selected from oxygen, nitrogen and sulfur;
   or a pharmaceutically acceptable salt thereof; or an optical isomer thereof; or a mixture of optical isomers thereof.

Preferred are the compounds in the B group, wherein
R is hydrogen, chloro, n-propyl or methoxy;
   or a pharmaceutically acceptable salt thereof; or an optical isomer thereof; or a mixture of optical isomers thereof.

Further preferred are also the compounds of formula (IB), designated as the C group, wherein
R₁ is hydrogen or optionally substituted alkyl;
R is hydrogen, halogen, optionally substituted C₁₋₆alkyl or C₁₋₆alkoxy;
Z is a bond, O or S;
p is an integer from 2-3;
Q is O or S;
W is aryl or heterocyclyl;
   or a pharmaceutically acceptable salt thereof; or an optical isomer thereof; or a mixture of optical isomers thereof.

Preferred are the compounds in the C group, wherein
R is hydrogen, chloro, n-propyl or methoxy;
   or a pharmaceutically acceptable salt thereof; or an optical isomer thereof; or a mixture of optical isomers thereof.

Another preferred group of compounds in the C group are the compounds, wherein W is selected from the group consisting of

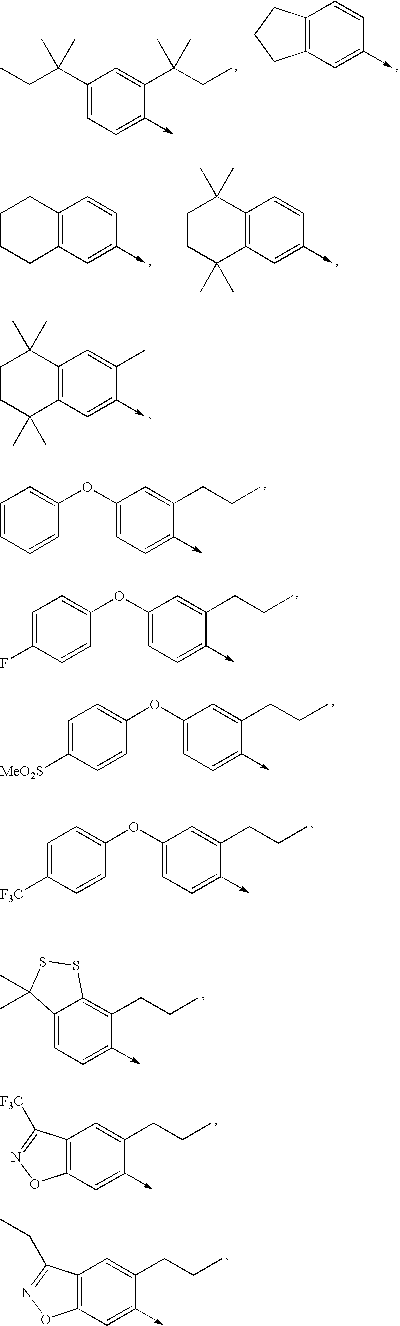

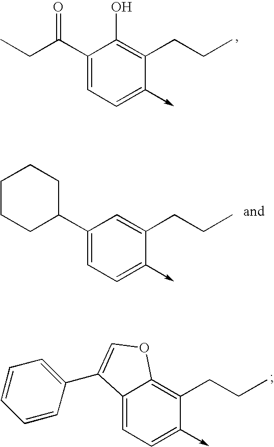

or a pharmaceutically acceptable salt thereof; or an optical isomer thereof; or a mixture of optical isomers thereof.

Further preferred are also the compounds of formula (IB), designated as the D group, wherein
R₁ is hydrogen or optionally substituted alkyl;
R is hydrogen, halogen, optionally substituted C₁₋₆alkyl or C₁₋₆alkoxy;
Z is O or S;
p is an integer from 1-2;
Q is a bond;
W is aryl or heterocyclyl;
   or a pharmaceutically acceptable salt thereof; or an optical isomer or a mixture of optical isomers thereof.

Preferred are the compounds in the D group, wherein
R is hydrogen, chloro, n-propyl or methoxy;
   or a pharmaceutically acceptable salt thereof; or an optical isomer thereof; or a mixture of optical isomers thereof.

Another preferred group of compounds in the D group are the compounds, wherein W is selected from the group consisting of

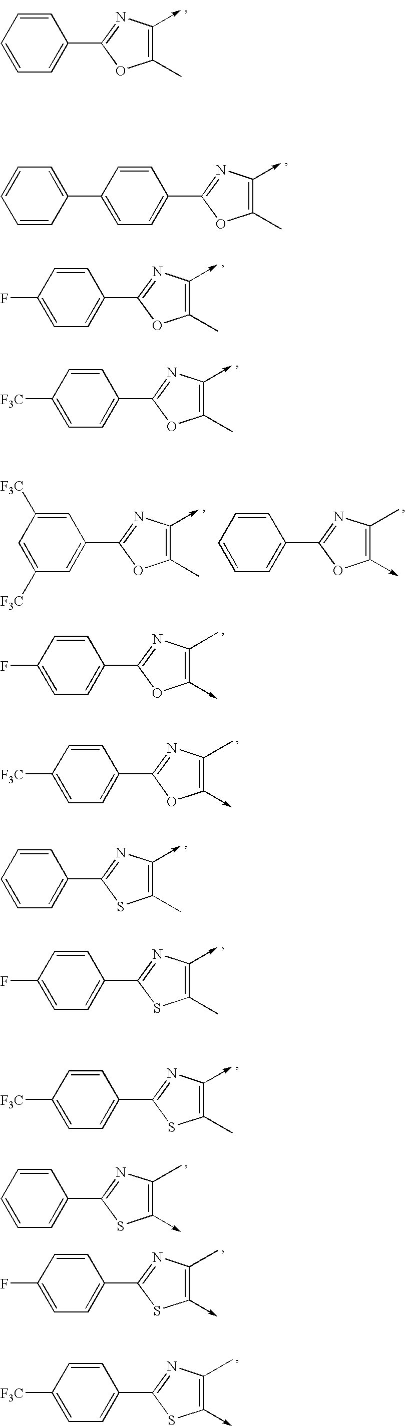

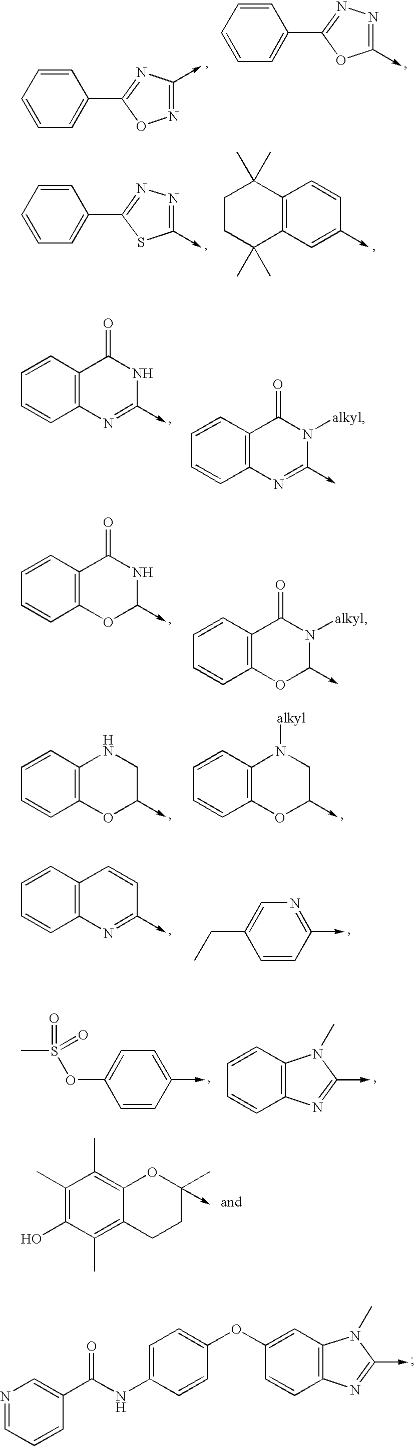

or a pharmaceutically acceptable salt thereof; or an optical isomer thereof; or a mixture of optical isomers thereof.

Yet another preferred group of compounds in the D group are also the compounds, wherein
R₁ is hydrogen or optionally substituted alkyl;
R is hydrogen, halogen, optionally substituted C₁₋₆ alkyl or C₁₋₆ alkoxy;
Z is O or S;
p is 2;
Q is a bond;
W is selected from the group consisting of

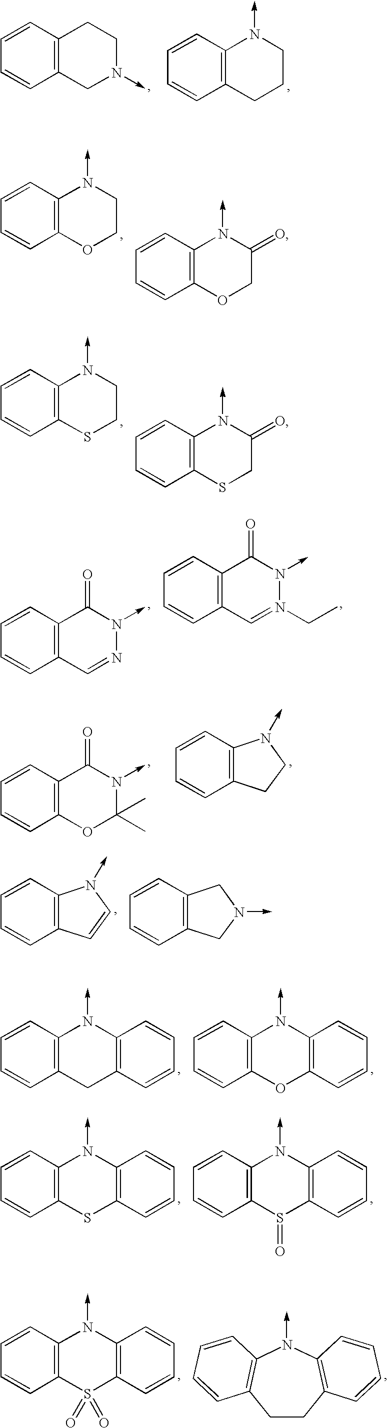

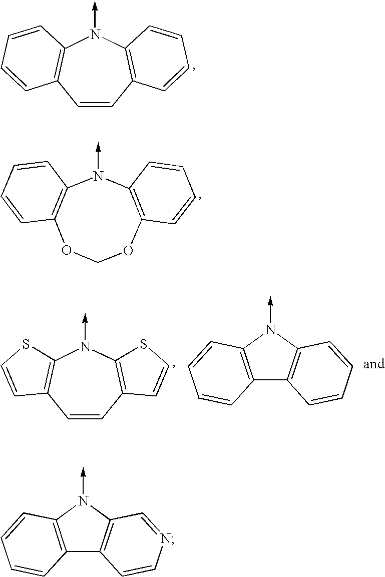

or a pharmaceutically acceptable salt thereof; or an optical isomer thereof; or a mixture of optical isomers thereof.

Particular embodiments of the invention are:
(R)-1-{4-[4-(4-Phenoxy-2-propyl-phenoxy)-butoxy]-benzenesulfonyl}-azetidine-2-carboxylic acid;
(R)-1-{4-[3-(4-Phenoxy-2-propyl-phenoxy)-propoxy]-benzenesulfonyl}-azetidine-2-carboxylic acid;
(R)-1-[4-(5-Methyl-2-phenyl-oxazol-4-ylmethoxy)-benzenesulfonyl]-azetidine-2-carboxylic acid;
(R)-1-{4-[2-(4-Fluoro-phenyl)-5-methyl-oxazol-4-ylmethoxy]-benzenesulfonyl}-azetidine-2-carboxylic acid;
(R)-1-{4-[5-Methyl-2-(4-trifluoromethyl-phenyl)-oxazol-4-ylmethoxy]-benzenesulfonyl}-azetidine-2-carboxylic acid;
(R)-1-{4-[2-(3,5-bis-Trifluoromethyl-phenyl)-5-methyl-oxazol-4-ylmethoxy]-benzene-sulfonyl}-azetidine-2-carboxylic acid;
(R)-1-{4-[2-(5-Methyl-2-phenyl-oxazol-4-yl)-ethoxy]-benzenesulfonyl}-azetidine-2-carboxylic acid;
(R)-1-{4-[4-(4-Phenoxy-2-propyl-phenoxy)-butoxy]-benzenesulfonyl}-pyrrolidine-2-carboxylic acid;
(R)-1-{4-[3-(4-Phenoxy-2-propyl-phenoxy)-propoxy]-benzenesulfonyl}-pyrrolidine-2-carboxylic acid;
(R)-1-(4-{3-[2-Propyl-4-(4-trifluoromethyl-phenoxy)-phenoxy]-propoxy}-benzenesulfonyl)-pyrrolidine-2-carboxylic acid;
(R)-1-{4-[2-(4-Phenoxy-2-propyl-phenoxy)-ethoxy]-benzenesulfonyl}-pyrrolidine-2-carboxylic acid;
(R)-1-(4-{2-[2-Propyl-4-(4-trifluoromethyl-phenoxy)-phenoxy]-ethoxy)-benzenesufonyl}-pyrrolidine-2-carboxylic acid;
(R)-1-{3-Methoxy-4-[3-(4-phenoxy-2-propyl-phenoxy)-propoxy]-benzenesulfonyl}-pyrrolidine-2-carboxylic acid;
(R)-1-{3-Chloro-4-[3-(4-phenoxy-2-propyl-phenoxy)-propoxy]-benzenesulfonyl}-pyrrolidine-2-carboxylic acid;
(R)-1-{4-[3-(4-Phenoxy-2-propyl-phenoxy)-propoxy]-3-propyl-benzenesulfonyl}-pyrrolidine-2-carboxylic acid;
(R)-1-{4-[3-(4-Phenoxy-2-propyl-phenoxy)-propylsulfanyl]-benzenesulfonyl}-pyrrolidine-2-carboxylic acid;
(R)-1-{4-[2-(4-Phenoxy-2-propyl-phenoxy)-ethylsulfanyl]-benzenesulfonyl}-pyrrolidine-2-carboxylic acid;
(R)-1-{4-[3-(4-Phenoxy-2-propyl-phenoxy)-propyl]-benzenesulfonyl}-pyrrolidine-2-carboxylic acid;
(R)-1-[4-(5-Methyl-2-phenyl-oxazol-4-ylmethoxy)-benzenesulfonyl]-pyrrolidine-2-carboxylic acid;
(R)-1-{4-[2-(4-Fluoro-phenyl)-5-methyl-oxazol-4-ylmethoxy]-benzenesulfonyl}-pyrrolidine-2-carboxylic acid;
(R)-1-{4-[5-Methyl-2-(4-trifluoromethyl-phenyl)-oxazol-4-ylmethoxy]-benzenesulfonyl}-pyrrolidine-2-carboxylic acid;
(R)-1-{4-[2-(3,5-bis-Trifluoromethyl-phenyl)-5-methyl-oxazol-4-ylmethoxy]-benzene-sulfonyl}-pyrrolidine-2-carboxylic acid;
(R)-1-[4-(2-Biphenyl-4-yl-5-methyl-oxazol-4-ylmethoxy)-benzenesulfonyl]-pyrrolidine-2-carboxylic acid;
(R)-1-[3-Methoxy-4-(5-methyl-2-phenyl-oxazol-4-ylmethoxy)-benzenesulfonyl]-pyrrolidine-2-carboxylic acid;
(R)-1-[3-Chloro-4-(5-methyl-2-phenyl-oxazol-4-ylmethoxy)-benzenesulfonyl]-pyrrolidine-2-carboxylic acid;
(R)-1-[4-(5-Methyl-2-phenyl-oxazol-4-ylmethoxy)-3-propyl-benzenesulfonyl]-pyrrolidine-2-carboxylic acid;
(R)-1-[4-(5-Methyl-2-phenyl-oxazol-4-ylmethylsulfanyl)-benzenesulfonyl]-pyrrolidine-2-carboxylic acid;
(R)-1-{4-[2-(4-Fluoro-phenyl)-5-methyl-oxazol-4-ylmethylsulfanyl]-benzenesulfonyl}-pyrrolidine-2-carboxylic acid;
(R)-1-{4-[5-Methyl-2-(4-trifluoromethyl-phenyl)-oxazol-4-ylmethylsulfanyl]-benzenesulfonyl}-pyrrolidine-2-carboxylic acid;
(R)-1-{4-[2-(3,5-bis-Trifluoromethyl-phenyl)-5-methyl-oxazol-4-ylmethylsulfanyl]-benzenesulfonyl}-pyrrolidine-2-carboxylic acid;
(R)-1-{4-[2-(5-Methyl-2-phenyl-oxazol-4-yl)-ethoxy]-benzenesulfonyl}-pyrrolidine-2-carboxylic acid;
(R)-1-{3-Methoxy-4-[2-(5-methyl-2-phenyl-oxazol-4-yl)-ethoxy]-benzenesulfonyl}-pyrrolidine-2-carboxylic acid;
(R)-1-{3-Chloro-4-[2-(5-methyl-2-phenyl-oxazol-4-yl)-ethoxy]-benzenesulfonyl}-pyrrolidine-2-carboxylic acid;
(R)-1-(4-{2-[5-Methyl-2-(4-trifluoromethyl-phenyl)-oxazol-4-yl]-ethoxy}-benzenesulfonyl)-pyrrolidine-2-carboxylic acid;
(R)-1-{4-[2-(5-Methyl-2-phenyl-oxazol-4-yl)-ethylsulfanyl]-benzenesulfonyl}-pyrrolidine-2-carboxylic acid;
(R)-1-{4-[4-(4-Phenoxy-2-propyl-phenoxy)-butoxy]-benzenesulfonyl}-2,3-dihydro-1H-indole-2-carboxylic acid;
(R)-1-{4-[3-(4-Phenoxy-2-propyl-phenoxy)-propoxy]-benzenesulfonyl}-2,3-dihydro-1H-indole-2-carboxylic acid;
(R)-1-{4-[2-(4-Phenoxy-2-propyl-phenoxy)-ethoxy]-benzenesulfonyl}-2,3-dihydro-1H-indole-2-carboxylic acid;
(R)-1-{3-Methoxy-4-[3-(4-phenoxy-2-propyl-phenoxy)-propoxy]-benzenesulfonyl}-2,3-dihydro-1H-indole-2-carboxylic acid;
(R)-1-{3-Chloro-4-[3-(4-phenoxy-2-propyl-phenoxy)-propoxy]-benzenesulfonyl}-2,3-dihydro-1H-indole-2-carboxylic acid;
(R)-1-[4-(5-Methyl-2-phenyl-oxazol-4-ylmethoxy)-benzenesulfonyl]-2,3-dihydro-1H-indole-2-carboxylic acid;
(R)-1-{4-[2-(4-Fluoro-phenyl)-5-methyl-oxazol-4-ylmethoxy]-benzenesulfonyl}-2,3-dihydro-1H-indole-2-carboxylic acid;
(R)-1-{4-[5-Methyl-2-(4-trifluoromethyl-phenyl)-oxazol-4-ylmethoxy]-benzenesulfonyl}-2,3-dihydro-1H-indole-2-carboxylic acid;
(R)-1-{4-[2-(3,5-bis-Trifluoromethyl-phenyl)-5-methyl-oxazol-4-ylmethoxy]-benzene-sulfonyl}-2,3-dihydro-1H-indole-2-carboxylic acid;
(R)-1-[3-Methoxy-4-(5-methyl-2-phenyl-oxazol-4-ylmethoxy)-benzenesulfonyl]-2,3-dihydro-1H-indole-2-carboxylic acid;
(R)-1-[3-Chloro-4-(5-methyl-2-phenyl-oxazol-4-ylmethoxy)-benzenesulfonyl]-2,3-dihydro-1H-indole-2-carboxylic acid;
(R)-1-[4-(5-Methyl-2-phenyl-oxazol-4-ylmethoxy)-3-propyl-benzenesulfonyl]-2,3-dihydro-1H-indole-2-carboxylic acid;
(R)-1-[4-(5-Methyl-2-phenyl-oxazol-4-ylmethylsulfanyl)-benzenesulfonyl]-2,3-dihydro-1H-indole-2-carboxylic acid;
(R)-1-{4-[2-(4-Fluoro-phenyl)-5-methyl-oxazol-4-ylmethylsulfanyl]-benzenesulfonyl}-2,3-dihydro-1H-indole-2-carboxylic acid;
(R)-1-{4-[5-Methyl-2-(4-trifluoromethyl-phenyl)-oxazol-4-ylmethylsulfanyl]-benzenesulfonyl}-2,3-dihydro-1H-indole-2-carboxylic acid;
(R)-1-{4-[2-(3,5-bis-Trifluoromethyl-phenyl)-5-methyl-oxazol-4-ylmethylsulfanyl]-benzenesulfonyl}-2,3-dihydro-1H-indole-2-carboxylic acid;
(R)-1-{4-[2-(5-Methyl-2-phenyl-oxazol-4-yl)-ethoxy]-benzenesulfonyl}-2,3-dihydro-1H-indole-2-carboxylic acid;
(R)-1-{3-Chloro-4-[2-(5-methyl-2-phenyl-oxazol-4-yl)-ethoxy]-benzenesulfonyl}-2,3-dihydro-1H-indole-2-carboxylic acid;
(R)-1-{4-[5-Methyl-2-(4-trifluoromethyl-phenyl)-oxazol-4-ylmethylsulfanyl]-benzene-sulfonyl}-pyrrolidine-2-carboxylic acid; and
(R)-1-{4-[5-Methyl-2-(4-trifluoromethyl-phenyl)-oxazol-4-ylmethylsulfanyl]-benzene-sulfonyl}-2,3-dihydro-1H-indole-2-carboxylic acid;
   or a pharmaceutically acceptable salt thereof; or an enantiomer thereof; or a mixture of enantiomers thereof.

Pharmaceutically acceptable salts of any acidic compounds of the invention are salts formed with bases, namely cationic salts, such as alkali; and alkaline earth metal salts, such as sodium, lithium, potassium, calcium and magnesium; as well as ammonium salts, such as ammonium, trimethylammonium, diethylammonium and tris-(hydroxymethyl)-methyl-ammonium salts.

Similarly acid addition salts, such as of mineral acids, organic carboxylic and organic sulfonic acids, e.g., hydrochloric acid, methanesulfonic acid and maleic acid, are possible provided a basic group, such as pyridyl, constitutes part of the structure.

The dual PPARα/γ agonist compounds of the present invention may be prepared as described in co-owned pending U.S. application Ser. No. 10/495,992, filed Nov. 20, 2002, herein incorporated herein by reference in its entirety as if set forth in full herein.

Depending on the choice of starting materials and methods, the compounds may be in the form of one of the possible isomers or mixtures thereof, e.g., as substantially pure geometric (cis or trans) isomers, optical isomers (antipodes), racemates or mixtures thereof. The aforesaid possible isomers or mixtures thereof are within the purview of this invention.

Any resulting mixtures of isomers can be separated on the basis of the physico-chemical differences of the constituents, into the pure geometric or optical isomers, diastereoisomers, racemates, e.g., by chromatography and/or fractional crystallization.

Any resulting racemates of final products or intermediates can be resolved into the optical antipodes by known methods, e.g., by separation of the diastereoisomeric salts thereof, obtained with an optically active acid or base, and liberating the optically active acidic or basic compound. The carboxylic acid intermediates can thus be resolved into their optical antipodes, e.g., by fractional crystallization of D- or L-(α-methylbenzylamine, cinchonidine, cinchonine, quinine, quinidine, ephedrine, dehydroabietylamine, brucine or strychnine)-salts. Racemic products can also be resolved by chiral chromatography, e.g., high-pressure liquid chromatography using a chiral adsorbent.

Finally, compounds of the invention are either obtained in the free form, or as a salt thereof if salt forming groups are present.

Acidic compounds of the invention may be converted into salts with pharmaceutically acceptable bases, e.g., an aqueous alkali metal hydroxide, advantageously in the presence of an ethereal or alcoholic solvent, such as a lower alkanol. From the solutions of the latter, the salts may be precipitated with ethers, e.g., diethyl ether. Resulting salts may be converted into the free compounds by treatment with acids. These or other salts can also be used for purification of the compounds obtained.

Compounds of the invention having basic groups can be converted into acid addition salts, especially pharmaceutically acceptable salts. These are formed, e.g., with inorganic acids, such as mineral acids, e.g., sulfuric acid, a phosphoric or hydrohalic acid; or with organic carboxylic acids, such as C₁₋₄alkanecarboxylic acids which, e.g., are unsubstituted or substituted by halogen, e.g., acetic acid, such as saturated or unsaturated dicarboxylic acids, e.g., oxalic, succinic, maleic or fumaric acid, such as hydroxy-carboxylic acids, e.g., glycolic, lactic, malic, tartaric or citric acid, such as amino acids, e.g., aspartic or glutamic acid; or with organic sulfonic acids, such as C₁₋₄alkyl-sulfonic acids, e.g., methanesulfonic acid; or arylsulfonic acids which are unsubstituted or substituted, e.g., by halogen. Preferred are salts formed with hydrochloric acid, methanesulfonic acid and maleic acid.

In view of the close relationship between the free compounds and the compounds in the form of their salts, whenever a compound is referred to in this context, a corresponding salt is also intended, provided such is possible or appropriate under the circumstances.

The compounds, including their salts, can also be obtained in the form of their hydrates, or include other solvents used for their crystallization.

The pharmaceutical compositions according to the invention are those suitable for enteral, such as oral or rectal, transdermal and parenteral administration to mammals, including man, for the treatment and/or prevention of conditions mediated by PPAR receptors, in particular, PPARα and PPARγ. Such conditions include those conditions mentioned hereinafter with respect to the treatment for which the compounds of the instant invention may be employed. The said pharmaceutical compositions comprise an effective amount of a pharmacologically active compound of the instant invention, alone or in combination with one or more pharmaceutically acceptable carriers.

The pharmacologically active compounds of the invention may be employed in the manufacture of pharmaceutical compositions comprising an effective amount thereof in conjunction or admixture with excipients or carriers suitable for either enteral or parenteral application. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, preferably about 1-50%, of the active ingredient.

Suitable formulations for transdermal application include a therapeutically effective amount of a compound of the invention with carrier. The term pharmaceutically effective amount as used herein indicates an amount necessary to administer to a host to achieve a therapeutic result, especially an inhibitory effect on end-organ damage, particularly to the heart and kidney. Advantageous carriers include absorbable pharmacologically acceptable solvents to assist passage through the skin of the host. Characteristically, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

A unit dosage for a mammal of about 50-70 kg may contain between about 1 μg and 1,000 μg, advantageously between about 5-500 μg of the active ingredient. The therapeutically effective dosage of active compound is dependent on the species of warm-blooded animal (mammal), the body weight, age and individual condition, on the form of administration and on the compound involved.

In another aspect of the present invention, there is provided a drug delivery device for local administration of a therapeutically effective amount of a dual PPARα/γ agonist compound or a pharmaceutically acceptable salt thereof or pharmaceutically acceptable salts thereof for the treatment and/or prevention of VSMC proliferative diseases or disorders.

A local delivery device or system according to the invention can be used to deliver the dual PPARα/γ compounds of the present invention for treatment of and/or prevention of stabilizing vulnerable plaques in arterial vessels, arterio-venous vascular access dysfunction in association with the insertion or repair of an indwelling shunt, fistula or catheter, arterial or venous aneurisms, anastomic hyperplasia, and arterial, e.g., aortic; and by-pass anastomosis.

The local administration preferably takes place at or near the lesion sites, e.g., vascular lesion sites.

The local administration may be by one or more of the following routes: via catheter or other intravascular delivery system, intranasally, intrabronchially, interperitoneally or esophageal, or via delivery balloons used in the musculature, e.g., left ventricle. Hollow tubes include natural body vessels or ducts, e.g., circulatory system vessels, such as blood vessels (arteries or veins, such as coronary, peripheral, renal or carotid arteries); tissue lumen; lymphatic pathways; digestive tract including alimentary duct, e.g., esophagus or biliary ducts; respiratory tract, e.g., trachea; excretory system tubes, e.g., intestines, ureters or urethra-prostate; reproductive system tubes and ducts, body cavity tubes, etc. Local administration or application of the compound(s) of the present invention may afford concentrated delivery of said compound(s), achieving tissue levels in target tissues not otherwise obtainable through other administration routes. Additionally local administration or application may reduce the risk of remote or systemic toxicity. Preferably, smooth muscle cell proliferation or migration is inhibited or reduced according to the invention immediately proximal or distal to the locally treated or stented area.

Means for local delivery of the compound(s) to hollow tubes can be by physical delivery of the compound(s) either internally or externally to the hollow tube. Local compound(s) delivery includes catheter delivery systems, local injection devices or systems or in-dwelling devices. Such devices or systems would include, but not be limited to, stents; coated stents; endolumenal sleeves; stent-grafts; sheathes; balloons; liposomes; controlled-release matrices; polymeric endoluminal paving; or other endovascular devices; embolic delivery particles; cell targeting, such as affinity based delivery; internal patches around the hollow tube, external patches around the hollow tube; hollow tube cuff; external paving; external stent sleeves; and the like. See Eccleston et al., Interventional Cardiol Monitor, Vol. 1, 33-40-41 (1995), Slepian, Interventional Cardiol, Vol. 1, pp. 103-116 (1996); or Regar, Sianos and Serruys, Br Med Bull, Vol. 59, pp. 277-248 (2001), which disclosures are herein incorporated by reference. Preferred stents include coronary artery, carotid artery, renal, iliac, femoral, popliteal, tibial and visceral stents. Also preferred are periadventitial drug delivery devices, arterio-venous access grafts, inventricular drug releasing balloons, drug-eluting surgical wraps used in organ surgery, drug-eluting arterial-venous PTFE access grafts in renal hemodialysis and drug-eluting surgical meshes used in hernia repair.

Preferably the delivery device or system fulfills pharmacological, pharmacokinetic and mechanical requirements. Preferably it also is suitable for sterilization. The stents according to the invention can be any stent, including self-expanding stent, or a stent that is radially expandable by inflating a balloon or expanded by an expansion member, or a stent that is expanded by the use of radio frequency which provides heat to cause the stent to change its size. A stent composed of or coated with a polymer or other biocompatible materials, e.g., porous ceramic, e.g., nanoporous ceramic, into which the compound(s) has been impregnated or incorporated can be used. Stents can be biodegradable or can be made of metal or alloy including, but not limited to, Cr, Co, Ni and Ti, or another stable substance when intended for permanent use. The compound(s) may also be entrapped into the metal of the stent or graft body which has been modified to contain micropores or channels. Also lumenal and/or ablumenal coating or external sleeve made of polymer or other biocompatible materials, e.g., as disclosed below, that contain the compound(s) can also be used for local delivery.

By “biocompatible” is meant a material which elicits no or minimal negative tissue reaction including, e.g., thrombus formation and/or inflammation.

Stents may commonly be used as a tubular structure left inside the lumen of a duct or arterial blood vessel to relieve an obstruction. They may be inserted into the duct and/or blood vessel lumen in a non-expanded form and are then expanded autonomously (self-expanding stents) or with the aid of a second device in situ, e.g., a catheter-mounted angioplasty balloon which is inflated within the stenosed vessel or body passageway in order to shear and disrupt the obstructions associated with the wall components of the vessel and to obtain an enlarged lumen. Alternatively, stents being easily deformed at lower temperature to be inserted in the hollow tubes may be used: after deployment at site. Such stents recover their original shape and exert a retentive and gentle force on the internal wall of the hollow tubes, e.g., of the esophagus or trachea.

The PPAR compound(s), optionally in the presence of another therapeutic agent, may be incorporated into or affixed to the stent in a number of ways and utilizing any biocompatible materials; it may be incorporated into, e.g., a polymer or a polymeric matrix and sprayed onto the outer surface of the stent. A mixture of the compound(s) and the polymeric material may be prepared in a solvent or a mixture of solvents and applied to the surfaces of the stents also by dip-coating, brush coating and/or dip/spin coating, the solvent(s) being allowed to evaporate to leave a film with entrapped compound(s). In the case of stents where the compound(s) is delivered from micropores, struts or channels, a solution of a polymer may additionally be applied as an out layer to control the compound(s) release; alternatively, the therapeutic agent may be comprised in the micropores, struts or channels and the active co-agent may be incorporated in the out layer, or vice versa. The therapeutic agent may also be affixed in an inner layer of the stent and the active co-agent in an outer layer, or vice versa. The compound(s) may also be attached by a covalent bond, e.g., esters, amides or anhydrides, to the stent surface, involving chemical derivatization. The compound(s) may also be incorporated into a biocompatible porous ceramic coating, e.g., a nanoporous ceramic coating. The medical device of the invention is configured to release the active co-agent concurrent with or subsequent to the release of the therapeutic agent.

Examples of polymeric materials include hydrophilic, hydrophobic or biocompatible biodegradable materials, e.g., polycarboxylic acids; cellulosic polymers; starch; collagen; hyaluronic acid; gelatin; lactone-based polyesters or copolyesters, e.g., polylactide; polyglycolide; polylactide-glycolide; polycaprolactone; polycaprolactone-glycolide; poly(hydroxybutyrate); poly(hydroxyvalerate); polyhydroxy(butyrate-co-valerate); polyglycolide-co-trimethylene carbonate; poly(diaxanone); polyorthoesters; polyanhydrides; polyaminoacids; polysaccharides; polyphospoeters; polyphosphoester-urethane; polycyanoacrylates; polyphosphazenes; poly(ether-ester) copolymers, e.g., PEO-PLLA, fibrin; fibrinogen; or mixtures thereof; and biocompatible non-degrading materials, e.g., polyurethane; polyolefins; polyesters; polyamides; polycaprolactame; polyimide; polyvinyl chloride; polyvinyl methyl ether; polyvinyl alcohol or vinyl alcohol/olefin copolymers, e.g., vinyl alcohol/ethylene copolymers; polyacrylonitrile; polystyrene copolymers of vinyl monomers with olefins, e.g., styrene acrylonitrile copolymers, ethylene methyl methacrylate copolymers; polydimethylsiloxane; poly(ethylene-vinylacetate); acrylate based polymers or copolymers, e.g., polybutylmethacrylate, poly(hydroxyethyl methylmethacrylate); polyvinyl pyrrolidinone; fluorinated polymers, such as polytetrafluoethylene; cellulose esters, e.g., cellulose acetate, cellulose nitrate or cellulose propionate; or mixtures thereof.

When a polymeric matrix is used, it may comprise two layers, e.g., a base layer in which the compound(s) is/are incorporated, e.g., ethylene-co-vinylacetate and polybutylmethacrylate, and a top coat, e.g., polybutylmethacrylate, which is compound(s)-free and acts as a diffusion-control of the compound(s). Alternatively, the therapeutic agent may be comprised in the base layer and the active co-agent may be incorporated in the out layer, or vice versa. Total thickness of the polymeric matrix may be from about 1-20μ or greater.

According to the method of the invention or in the device or system of the invention, the compound(s) may elute passively, actively or under activation, e.g., light-activation.

The compound(s) elutes from the polymeric material or the stent over time and enters the surrounding tissue, e.g., up to ca. 1-3 months. The local delivery according to the present invention allows for high concentration of the compound(s) at the disease site with low concentration of circulating compound. The amount of compound(s) used for local delivery applications will vary depending on the compounds used, the condition to be treated and the desired effect. For purposes of the invention, a therapeutically effective amount will be administered; e.g., the drug delivery device or system is configured to release the therapeutic agent and/or the active co-agent at a rate of 1-5,000 μg for a duration up to 60 days, preferably 10-500 μg, even more preferably 50-400 μg as an initial release within the first 48 hours following implantation followed by a release of 100-300 μg for up to 60 days or the depletion of the releasable drug, whichever comes first. By therapeutically effective amount is intended an amount sufficient to inhibit cellular proliferation and resulting in the prevention and treatment of the disease state. Specifically, for the prevention or treatment of restenosis in diabetics and non-diabetics, e.g., after revascularization, or anti-tumor treatment, local delivery will require less compound than systemic administration.

A contemplated treatment period as defined as the duration of drug release from the device for use in the prevention or reduction of vascular access dysfunction of the present invention is approximately 60 days at maximum, e.g., 45 days, preferably 28 days in association with the insertion or repair of an indwelling shunt, fistula or catheter, or actual treatment. In the case of the use of biodegradable stents or other devices for use in the prevention or reduction of vascular access dysfunction of the present invention the treatment period would similarly be is approximately 60 days at maximum, e.g., 45 days, preferably 28 days. However, the treatment period would be of less duration than that required for the complete degradation of the implanted biodegradable device.

In another aspect of the present invention, there is provided a method treating and/or preventing VSMC proliferative diseases or disorders comprising administering a therapeutically effective amount of a compound of the invention as defined above, either alone or in a combination with another therapeutic agent, e.g., each at an effective therapeutic dose as reported in the art. Such therapeutic agents include anti-organ rejection drugs, such as rapamycin, picrolimus, everolimus, ABT 578 and tacrolimus; cell cycle inhibitors such as paclitaxel and everolimus; PDGF/tyrosine kinase inhibitors, such as imatinib also known as Glivec®; bisphosphonates, such as zoledronic acid also known as Zometa®; non-steroidal anti-inflammatory compounds, such as pimecrolimus also known as Elidel®; PKC 412; anti-inflammatory steroids, such as prednisone; estrogen; aldosterone receptor antagonists, such as epleronone and spironolactone; aldosterone synthase inhibitors, such as FAD286; VEGF inhibitors; matrix metalloproteinase (MMP) inhibitors, such as batimistat, marimistat, trocade, CGS 27023, RS 130830 or AG3340; chymase inhibitors; a compound stimulating the release of (NO) or a NO donor, e.g., diazeniumdiolates, S-nitrosothiols, mesoionic oxatriazoles, isosorbide dinitrate or a combination thereof, e.g., mononitrate and/or dinitrate; antioxidants, such as AGI-1067 and BO-653; narcotic analgesics; non-narcotic analgesics; heparin and heparinoid drugs; low molecular weight heparin, such as enoxaparin and pentasaccharides; direct thrombin inhibitors; factor Xa inhibitors; factor Vila inhibitors; glycoprotein 2B/3A inhibitors (GP2B/3A); fibrinolytics, such as r-tPA; streptokinase; urokinase; desmeloptase; PAI-1 inhibitors; acyl-CoA:cholesterol acyl-transferase (ACAT) inhibitors such as eflucimibe; inhibitors of lipoprotein-associated phospholipase A2 (Lp-PLA2), such as SB-480848; 3-hydroxy-3-methyl-glutaryl coenzyme A (HMG-CoA) reductase inhibitors, such as fluvastatin, lovastatin, simvastatin, pravastatin, atorvastatin, cerivastatin, pitavastatin, rosuvastatin or nivastatin; cholesterol ester transferase inhibitors (CETPi), fibrinectin inhibitors, vitronectin inhibitors, platelet purinoceptor antagonists, such as ticlopidine, clopidogrel and MCP1 inhibitors.

A compound of the present invention may be administered either simultaneously, before or after the other active ingredient, either separately by the same or different route of administration or together with another therapeutic agent in the same pharmaceutical formulation.

Another aspect of the present invention relates to methods for the treatment and/or prevention of VSMC proliferative diseases or disorders, such as ureteral and/or biliary proliferation; and coronary artery and peripheral arterial stenosis; restenosis in diabetics and non-diabetics; inflammatory disorders, e.g., T-cell induced inflammation; stabilizing vulnerable plaques in blood vessels; vascular access dysfunction in association with the insertion or repair of an indwelling shunt fistula or catheter; arterial or venous aneurisms; anastomic hyperplasia; and arterial, e.g., aortic, by-pass anastomosis comprising administering the PPAR compound(s) of the present invention, either alone or in conjunction with another therapeutic agent, for the treatment and/or prevention of the VSMC proliferative diseases or disorders mentioned herein. A preferred method is a method for treating and/or preventing restenosis in a diabetic patient.

The above-cited properties are demonstrable in in vivo tests, using advantageously mammals, e.g., rats, dogs, monkeys, pigs or isolated organs, tissues and preparations thereof. Said compounds can be applied in vivo either enterally, parenterally or locally. A therapeutically effective amount in vivo may range depending on the route of administration, between about 1 mg/kg and 500 mg/kg, preferably between about 5 mg/kg and 100 mg/kg.

Diabetic animal models may be employed for the in vivo test, e.g., swine could be made diabetic as per the protocol of Larsen et al., Am J Physiol Endocrinol Metab, Vol. 282, pp. E1342-E1351 (2002); Fricker, DD, Vol. 6, No. 18, pp. 921-922 (2001); and Gerrity et al., Diabetes, Vol. 50, pp. 1654-1665 (2001).

The activity of a compound according to the invention can be assessed by the following methods or methods well-described in the art:

The treatment and/or prevention of restenosis in vivo in diabetics and non-diabetics can be evaluated as follows:
1. The beneficial effects of a compound of the present invention on restenosis are ascertained in a swine model of restenosis. Stainless steel balloon-expandable tubular stents, 18 mm long, are coated with a thin layer of a polymer, i.e., poly-n-butyl methacrylate, containing 200 mg of (R)-1-{4-[5-methyl-2-(4-trifluoromethyl-phenyl)-oxazol-4-ylmethoxy]-benzenesulfonyl}-2,3-dihydro-1H-indole-2-carboxylic acid (Compound A) per stent. Juvenile swine (25-35 kg) undergo placement of bare metal stents which elute Compound A in the left anterior descending, circumflex or right coronary artery. The guiding catheter is used as a reference to obtain a 1.2:1 to 1.4:1 stent-to-artery ratio compared with the baselinevessel diameter. Animals are allowed to recover and are returned to care facilities, where they receive a normal diet, aspirin 325 mg/d, and ticlopidine (250 mg/day) or clopidogrel (75 mg/day). At 7 days or 28 days, the animals are euthanized after completion of coronary angiography for quantitative analysis.
Immediately following euthanasia, the hearts are harvested, and the coronary arteries are perfusion-fixed with 10% buffered formalin at 60-80 mm Hg for 30 minutes via the aortic stump. The vessels from the 7-day group placement are dissected from the heart after perfusion with lactated Ringer's solution, cleaned of excess perivascular tissue and frozen in liquid nitrogen. Vessel wall expression of proliferating cell nuclear antigen (PCNA) (Santa Cruz Biotechnology), pRb (Pharmingen), monocyte chemotactic protein (MCP)-1 (R&D Systems) or interleukin (IL)-6 (R&D Systems) were evaluated by Western blot analysis. In the 28-day studies, the stented coronary artery segments are processed for plastic embedding, staining and histomorphometric analysis of 6 sections from the proximal aspect through the distal margin of the stent. A grading scheme is employed to assess arterial wall (collagen deposition, fibrin deposition, etc.) and cellular parameters, i.e., re-endotheliazation, VSMC proliferation, macrophage infiltration, etc., that determines the maturity of vascular repair.
The stent endothelialization score is defined as the extent of the circumference of the arterial lumen covered by endothelial cells and is scored from 1-3 (1=25%; 2=25-75%; 3=>75%). The intimal fibrin content is graded as: 1, focal residual fibrin involving any portion of the artery and for moderate fibrin deposition adjacent to the strut involving <25% of the circumference of the artery; 2, moderate fibrin deposition involving >25% of the circumference of the artery or heavy deposition of fibrin adjacent to and between stent struts involving <25% of the circumference of the artery; or 3, heavy deposition of fibrin involving >25% of the circumference of the artery. The intimal SMC content is scored as: 1, sparse SMC density involving any portion of the artery and for moderate SMC infiltration less than the full thickness of the neointima involving <25% of the circumference of the artery; 2, moderate SMC infiltration less than the full thickness of the neointima involving >25% of the circumference of the artery or dense SMC content the full thickness of the neointima involving <25% of the circumference of the artery; or 3, dense SMC content the full thickness of the neointima involving >25% of the circumference of the artery.
The mean angiographic, histological, morphological and densitometric data for each stent are compared by analysis of variance (ANOVA) with post hoc analysis for multiple comparisons. Significance was established by a value of p<0.05. The data will be expressed as mean±SD.
In this model, the treatment with the stent containing Compound A results in a marked reduction in the extent of restenotic lesion and arterial stenosis.
2. The beneficial effects of Compound A in diabetic restenosis are ascertained in a diabetic swine model of restenosis. Juvenile swine (25-35 kg), made diabetic by injection of approximately 100 mg of streptozotocin for up to 3 days or until plasma glucose levels were greater than 300 mg/decilitier, are used in these studies.

The procedure of 1 above is followed with the diabetic swine. In this model, the treatment with Compound A results in a reduction in the extent of restenotic lesion formation and arterial stenosis.

The following Examples are intended to illustrate the invention and are not to be construed as being limitations thereon. Temperatures are given in degrees Centigrade. If not mentioned otherwise, all evaporations are performed under reduced pressure, preferably between about 15 mm Hg and 100 mm Hg (=20-133 mbar). The structure of final products, intermediates and starting materials is confirmed by standard analytical methods, e.g., microanalysis and spectroscopic characteristics, e.g., MS, IR and NMR. Abbreviations used are those conventional in the art.

### EXAMPLE 1

The stent is manufactured from medical 316LS stainless steel and is composed of a series of cylindrically oriented rings aligned along a common longitudinal axis. Each ring consists of 3 connecting bars and 6 expanding elements. The stent is premounted on a delivery system. The therapeutic agent, e.g., (R)-1-{4-[5-methyl-2-(4-trifluoromethyl-phenyl)-oxazol-4-ylmethoxy]-benzenesulfonyl}-2,3-dihydro-1H-indole-2-carboxylic acid (0.50 mg/mL) is incorporated into a polymer matrix based on a semicrystalline ethylene-vinyl alcohol copolymer. The stent is coated with this matrix.

### EXAMPLE 2

A stent is weighed and then mounted for coating. While the stent is rotating, a solution of polylactide g lycolide, (R)-1-{4-[5-methyl-2-(4-trifluoromethyl-phenyl)-oxazol-4-ylmethoxy]-benzenesulfonyl}-2,3-dihydro-1H-indole-2-carboxylic acid, 0.0015 mg/mL 2,6-di-tert-butyl-4-methylphenol and 1 mg/mL tyrosine kinase C inhibitor dissolved in a mixture of methanol and tetrahydrofuran, is sprayed onto it. The coated stent is removed from the spray and allowed to air-dry. After a final weighing, the amount of coating on the stent is determined.

The tyrosine kinase C inhibitor may be replaced by taxol, paclitaxel, a VEGF receptor tyrosine kinase inhibitor, a VEGF receptor inhibitor, a compound binding to VEGF or an aldosterone receptor blocker, an aldosterone sytnase inhibitor, a compound inhibiting the renin-angiotensin system or an anti-inflammatory compound.

### EXAMPLE 3

Four 2 cm pieces of coated stents as described above are placed into 100 mL of phosphate buffer solution (PBS) having a pH of 7.4. Another 4 pieces from each series are placed into 100 mL polyethylene glycol (PEG)/water solution (40/60 v/v, MW of PEG=400). The stent pieces are incubated at 37° C. in a shaker. The buffer and PEG solutions are changed daily and different assays are performed on the solution to determine the released (R)-1-{4-[5-methyl-2-(4-trifluoromethyl-phenyl)-oxazol-4-ylmethoxy]-benzenesulfonyl}-2,3-dihydro-1H-indole-2-carboxylic acid concentrations. Such assays can show a stable release of (R)-1-{4-[5-methyl-2-(4-trifluoromethyl-phenyl)-oxazol-4-ylmethoxy]-benzenesulfonyl}-2,3-dihydro-1H-indole-2-carboxylic acid from coated stents for more than 45 days. By the term “stable-release of (R)-1-{4-[5-methyl-2-(4-trifluoromethyl-phenyl)-oxazol-4-ylmethoxy]-benzenesulfonyl}-2,3-dihydro-1H-indole-2-carboxylic acid” is meant less than 10% of variation of the drug release. Controlled-release techniques used by a person skilled in the art allow an unexpected easy adaptation of the required drug release rate. Thus, by selecting appropriate amounts of reactants in the coating mixture it is possible to easily control the bioeffectiveness of the (R)-1-{4-[5-methyl-2-(4-trifluoromethyl-phenyl)-oxazol-4-ylmethoxy]-benzenesulfonyl}-2,3-dihydro-1H-indole-2-carboxylic acid coated stents.

Although the present invention has been described in considerable detail with reference to certain preferred versions thereof, other versions are possible without departing from the spirit and scope of the preferred versions contained herein. All references and Patents (U.S. and others) referred to herein are hereby incorporated by reference in their entirety as if set forth in full herein.

What is claimed is:

1. A method of treating and/or preventing vascular smooth muscle cell (VSMC) proliferative diseases or disorders comprising administering a therapeutically effective amount of a dual PPARα/γ agonist compound or a pharmaceutically acceptable salt thereof to a mammal in need thereof.

2. The method of claim 1, further comprising administering said compound in combination with a therapeutically effective amount of an additional therapeutic agent.

3. The method of claim 2, wherein the additional therapeutic agent is an anti-organ rejection drug, a cell cycle inhibitor, a PDGF/tyrosine kinase inhibitors, a bisphosphonate, an anti-inflammatory steroid, an aldosterone receptor antagonist, an aldosterone synthase inhibitor, a matrix metalloproteinase (MMP) inhibitor, a chymase inhibitors; a compound stimulating the release of (NO) or a NO donor, an antioxidant, a non-steroidal anti-inflammatory drug, a narcotic analgesic, a non-narcotic analgesic, heparin or a heparinoid drug, a direct thrombin inhibitor, a factor Xa inhibitor, a factor VIIa inhibitors, a glycoprotein 2B/3A inhibitors (GP2B/3A), a fibrinolytic, a PAI-1 inhibitor, an acyl-CoA;cholesterol acyltransferase (ACAT) inhibitor; a lipoprotein-associated phospholipase A2 (Lp-PLA2) inhibitor; a 3-hydroxy-3-methyl-glutaryl coenzyme A (HMG-CoA) reductase inhibitor, a cholesterol ester transferase inhibitors (CETPi), a fibrinectin inhibitors, a vitronectin inhibitor, a platelet purinoceptor antagonist or a MCP1 inhibitor.

4. The method of claim 1, wherein the dual α/γ PPAR agonist is selected from the group consisting of:
(R)-1-{4-[4-(4-Phenoxy-2-propyl-phenoxy)-butoxy]-benzenesulfonyl}-azetidine-2-carboxylic acid;
(R)-1-{4-[3-(4-Phenoxy-2-propyl-phenoxy)-propoxy]-benzenesulfonyl}-azetidine-2-carboxylic acid;
(R)-1-[4-(5-Methyl-2-phenyl-oxazol-4-ylmethoxy)-benzenesulfonyl]-azetidine-2-carboxylic acid;
(R)-1-{4-[2-(4-Fluoro-phenyl)-5-methyl-oxazol-4-ylmethoxy]-benzenesulfonyl}-azetidine-2-carboxylic acid;
(R)-1-{4-[5-Methyl-2-(4-trifluoromethyl-phenyl)-oxazol-4-ylmethoxy]-benzenesulfonyl}-azetidine-2-carboxylic acid;
(R)-1-{4-[2-(3,5-bis-Trifluoromethyl-phenyl)-5-methyl-oxazol-4-ylmethoxy]-benzene-sulfonyl}-azetidine-2-carboxylic acid;
(R)-1-{4-[2-(5-Methyl-2-phenyl-oxazol-4-yl)-ethoxy]-benzenesulfonyl}-azetidine-2-carboxylic acid;
(R)-1-{4-[4-(4-Phenoxy-2-propyl-phenoxy)-butoxy]-benzenesulfonyl}-pyrrolidine-2-carboxylic acid;
(R)-1-{4-[3-(4-Phenoxy-2-propyl-phenoxy)-propoxy]-benzenesulfonyl}-pyrrolidine-2-carboxylic acid;
(R)-1-(4-{3-[2-Propyl-4-(4-trifluoromethyl-phenoxy)-phenoxy]-propoxy}-benzenesulfonyl)-pyrrolidine-2-carboxylic acid;
(R)-1-{4-[2-(4-Phenoxy-2-propyl-phenoxy)-ethoxy]-benzenesulfonyl}-pyrrolidine-2-carboxylic acid;
(R)-1-(4-{2-[2-Propyl-4-(4-trifluoromethyl-phenoxy)-phenoxy]-ethoxy}-benzenesulfonyl)-pyrrolidine-2-carboxylic acid;
(R)-1-{3-Methoxy-4-[3-(4-phenoxy-2-propyl-phenoxy)-propoxy]-benzenesulfonyl}-pyrrolidine-2-carboxylic acid;
(R)-1-{3-Chloro-4-[3-(4-phenoxy-2-propyl-phenoxy)-propoxy]-benzenesulfonyl}-pyrrolidine-2-carboxylic acid;
(R)-1-{4-[3-(4-Phenoxy-2-propyl-phenoxy)-propoxy]-3-propyl-benzenesulfonyl}-pyrrolidine-2-carboxylic acid;
(R)-1-{4-[3-(4-Phenoxy-2-propyl-phenoxy)-propylsulfanyl]-benzenesulfonyl}-pyrrolidine-2-carboxylic acid;
(R)-1-{4-[2-(4-Phenoxy-2-propyl-phenoxy)-ethylsulfanyl]-benzenesulfonyl}-pyrrolidine-2-carboxylic acid;
(R)-1-{4-[3-(4-Phenoxy-2-propyl-phenoxy)-propyl]-benzenesulfonyl}-pyrrolidine-2-carboxylic acid;
(R)-1-[4-(5-Methyl-2-phenyl-oxazol-4-ylmethoxy)-benzenesulfonyl]-pyrrolidine-2-carboxylic acid;
(R)-1-{4-[2-(4-Fluoro-phenyl)-5-methyl-oxazol-4-ylmethoxy]-benzenesulfonyl}-pyrrolidine-2-carboxylic acid;
(R)-1-{4-[5-Methyl-2-(4-trifluoromethyl-phenyl)-oxazol-4-ylmethoxy]-benzenesulfonyl}-pyrrolidine-2-carboxylic acid;
(R)-1-{4-[2-(3,5-bis-Trifluoromethyl-phenyl)-5-methyl-oxazol-4-ylmethoxy]-benzene-sulfonyl}-pyrrolidine-2-carboxylic acid;
(R)-1-[4-(2-Biphenyl-4-yl-5-methyl-oxazol-4-ylmethoxy)-benzenesulfonyl]-pyrrolidine-2-carboxylic acid;
(R)-1-[3-Methoxy-4-(5-methyl-2-phenyl-oxazol-4-ylmethoxy)-benzenesulfonyl]-pyrrolidine-2-carboxylic acid;
(R)-1-[3-Chloro-4-(5-methyl-2-phenyl-oxazol-4-ylmethoxy)-benzenesulfonyl]-pyrrolidine-2-carboxylic acid;
(R)-1-[4-(5-Methyl-2-phenyl-oxazol-4-ylmethoxy)-3-propyl-benzenesulfonyl]-pyrrolidine-2-carboxylic acid;
(R)-1-[4-(5-Methyl-2-phenyl-oxazol-4-ylmethylsulfanyl)-benzenesulfonyl]-pyrrolidine-2-carboxylic acid;
(R)-1-{4-[2-(4-Fluoro-phenyl)-5-methyl-oxazol-4-ylmethylsulfanyl]-benzenesulfonyl}-pyrrolidine-2-carboxylic acid;
(R)-1-{4-[5-Methyl-2-(4-trifluoromethyl-phenyl)-oxazol-4-ylmethylsulfanyl]-benzenesulfonyl}-pyrrolidine-2-carboxylic acid;
(R)-1-{4-[2-(3,5-bis-Trifluoromethyl-phenyl)-5-methyl-oxazol-4-ylmethylsulfanyl]-benzenesulfonyl}-pyrrolidine-2-carboxylic acid;
(R)-1-{4-[2-(5-Methyl-2-phenyl-oxazol-4-yl)-ethoxy]-benzenesulfonyl}-pyrrolidine-2-carboxylic acid;
(R)-1-{3-Methoxy-4-[2-(5-methyl-2-phenyl-oxazol-4-yl)-ethoxy]-benzenesulfonyl}-pyrrolidine-2-carboxylic acid;
(R)-1-{3-Chloro-4-[2-(5-methyl-2-phenyl-oxazol-4-yl)-ethoxy]-benzenesulfonyl}-pyrrolidine-2-carboxylic acid;
(R)-1-(4-{2-[5-Methyl-2-(4-trifluoromethyl-phenyl)-oxazol-4-yl]-ethoxy}-benzenesulfonyl)-pyrrolidine-2-carboxylic acid;
(R)-1-{4-[2-(5-Methyl-2-phenyl-oxazol-4-yl)-ethylsulfanyl]-benzenesulfonyl}-pyrrolidine-2-carboxylic acid;
(R)-1-{4-[4-(4-Phenoxy-2-propyl-phenoxy)-butoxy]-benzenesulfonyl}-2,3-dihydro-1H-indole-2-carboxylic acid;
(R)-1-{4-[3-(4-Phenoxy-2-propyl-phenoxy)-propoxy]-benzenesulfonyl}-2,3-dihydro-1H-indole-2-carboxylic acid;
(R)-1-{4-[2-(4-Phenoxy-2-propyl-phenoxy)-ethoxy]-benzenesulfonyl}-2,3-dihydro-1H-indole-2-carboxylic acid;
(R)-1-{3-Methoxy-4-[3-(4-phenoxy-2-propyl-phenoxy)-propoxy]-benzenesulfonyl}-2,3-dihydro-1H-indole-2-carboxylic acid;
(R)-1-{3-Chloro-4-[3-(4-phenoxy-2-propyl-phenoxy)-propoxy]-benzenesulfonyl}-2,3-dihydro-1H-indole-2-carboxylic acid;
(R)-1-[4-(5-Methyl-2-phenyl-oxazol-4-ylmethoxy)-benzenesulfonyl]-2,3-dihydro-1H-indole-2-carboxylic acid;
(R)-1-{4-[2-(4-Fluoro-phenyl)-5-methyl-oxazol-4-ylmethoxy]-benzenesulfonyl}-2,3-dihydro-1H-indole-2-carboxylic acid;
(R)-1-{4-[5-Methyl-2-(4-trifluoromethyl-phenyl)-oxazol-4-ylmethoxy]-benzenesulfonyl}-2,3-dihydro-1H-indole-2-carboxylic acid;
(R)-1-{4-[2-(3,5-bis-Trifluoromethyl-phenyl)-5-methyl-oxazol-4-ylmethoxy]-benzene-sulfonyl}-2,3-dihydro-1H-indole-2-carboxylic acid;
(R)-1-[3-Methoxy-4-(5-methyl-2-phenyl-oxazol-4-ylmethoxy)-benzenesulfonyl]-2,3-dihydro-1H-indole-2-carboxylic acid;
(R)-1-[3-Chloro-4-(5-methyl-2-phenyl-oxazol-4-ylmethoxy)-benzenesulfonyl]-2,3-dihydro-1H-indole-2-carboxylic acid;
(R)-1-[4-(5-Methyl-2-phenyl-oxazol-4-ylmethoxy)-3-propyl-benzenesulfonyl]-2,3-dihydro-1H-indole-2-carboxylic acid;
(R)-1-[4-(5-Methyl-2-phenyl-oxazol-4-ylmethylsulfanyl)-benzenesulfonyl]-2,3-dihydro-1H-indole-2-carboxylic acid;
(R)-1-{4-[2-(4-Fluoro-phenyl)-5-methyl-oxazol-4-ylmethylsulfanyl]-benzenesulfonyl}-2,3-dihydro-1H-indole-2-carboxylic acid;
(R)-1-{4-[5-Methyl-2-(4-trifluoromethyl-phenyl)-oxazol-4-ylmethylsulfanyl]-benzenesulfonyl}-2,3-dihydro-1H-indole-2-carboxylic acid;
(R)-1-{4-[2-(3,5-bis-Trifluoromethyl-phenyl)-5-methyl-oxazol-4-ylmethylsulfanyl]-benzenesulfonyl}-2,3-dihydro-1H-indole-2-carboxylic acid;
(R)-1-{4-[2-(5-Methyl-2-phenyl-oxazol-4-yl)-ethoxy]-benzenesulfonyl}-2,3-dihydro-1H-indole-2-carboxylic acid;
(R)-1-{3-Chloro-4-[2-(5-methyl-2-phenyl-oxazol-4-yl)-ethoxy]-benzenesulfonyl}-2,3-dihydro-1H-indole-2-carboxylic acid;
(R)-1-{4-[5-Methyl-2-(4-trifluoromethyl-phenyl)-oxazol-4-ylmethylsulfanyl]-benzene-sulfonyl}-pyrrolidine-2-carboxylic acid; and
(R)-1-{4-[5-Methyl-2-(4-trifluoromethyl-phenyl)-oxazol-4-ylmethylsulfanyl]-benzene-sulfonyl}-2,3-dihydro-1H-indole-2-carboxylic acid;
or a pharmaceutically acceptable salt thereof; or an enantiomer thereof; or a mixture of enantiomers thereof.

5. The method of claim 4, wherein the dual α/γ PPAR agonist is (R)-1-{4-[5-methyl-2-(4-trifluoromethyl-phenyl)-oxazol-4-ylmethoxy]-benzenesulfonyl}-2,3-dihydro-1H-indole-2-carboxylic acid.

6. The method of claim 5, wherein the VSMC proliferative disease or disorder is ureteral and/or biliary proliferation, stenosis, restenosis in diabetics and non-diabetics, inflammatory disorders, vulnerable plaques or vascular access dysfunction in association with the insertion or repair of an indwelling shunt, fistula or catheter, arterial or venous aneurisms, anastomic hyperplasia and arterial by-pass anastomosis.

7. The method of claim 6, wherein the stenosis is and coronary artery and peripheral arterial sclerosis.

8. The method of claim 6, wherein the VSMC proliferative disease or disorder is restenosis in a diabetic patient.

9. A drug delivery device for local administration comprising a therapeutically effective amount of a dual PPARα/γ agonist compound or a pharmaceutically acceptable salt thereof.

10. The drug delivery device of claim 9, wherein the device is a stent.

11. The device of claim 10, wherein the dual PPARα/γ agonist compound is (R)-1-{4-[5-methyl-2-(4-trifluoromethyl-phenyl)-oxazol-4-ylmethoxy]-benzenesulfonyl}-2,3-dihydro-1H-indole-2-carboxylic acid.

12. The drug delivery device of claim 9, further comprising administering said compound in combination with a therapeutically effective amount of an additional therapeutic agent.

13. The device of claim 12, wherein the additional therapeutic agent is an anti-organ rejection drug, a cell cycle inhibitor, PDGF/Tyrosine kinase inhibitors, a bisphosphonate, an anti-inflammatory steroid or non-steroid, an aldosterone receptor antagonist, an aldosterone synthase inhibitor, a MMP inhibitor, a chymase inhibitor; a compound stimulating the release of (NO) or a NO donor, an antioxidant, a non-steroidal anti-inflammatory drug, a narcotic analgesic, a non-narcotic analgesic, heparin or a heparinoid drug, a direct thrombin inhibitor, a factor Xa inhibitor, a factor VIIa inhibitor, a GP2B/3A, a fibrinolytic, a PAI-1 inhibitor, an ACAT inhibitor, a Lp-PLA2 inhibitor, a HMG-CoA reductase inhibitor, a cholesterol ester transferase protein inhibitor, a fibrinectin inhibitor, a vitronectin inhibitor, a platelet purinoceptor antagonist or an MCP1 inhibitor.

14. A method of treating and/or preventing VSMC proliferative diseases or disorders comprising administering the device of claim 11 to a mammal in need thereof.

15. The method of claim 14, wherein the VSMC proliferative disease or disorder is ureteral and/or biliary proliferation, stenosis, restenosis in diabetics and non-diabetics, inflammatory disorders, vulnerable plaques or vascular access dysfunction in association with the insertion or repair of an indwelling shunt, fistula or catheter, arterial or venous aneurisms, anastomic hyperplasia and arterial by-pass anastomosis.

16. The method of claim 15, wherein the stenosis is and coronary artery and peripheral arterial sclerosis.

17. The method of claim 15, wherein the VSMC proliferative disease or disorder is restenosis in a diabetic patient.
